(19) United States
(12) Patent Application Publication
(10) Pub. No.: US 2009/0264755 A1
(43) Pub. Date: Oct. 22, 2009

(54) High-Intensity Ultrasonic Vessel Ablator Using Blood Flow Signal for Precise Positioning

(75) Inventors: Wen-Shiang Chen, Taipei (TW); Chih-Ching Wu, Taoyuan County (TW); Chiung-Nien Chen, Taipei City (TW); Ming-Chih Ho, Taipei City (TW); Po-Huang Lee, Taipei City (TW)

Correspondence Address: WPAT, PC;INTELLECTUAL PROPERTY ATTORNEYS, 2030 MAIN STREET, SUITE 1300, IRVINE, CA, 92614, US

(73) Assignee: National Taiwan University, Taipei City (TW)

(21) Appl. No.: 12/107,723

(22) Filed: Apr. 22, 2008

(51) Int. Cl. A61B 8/06; A61N 7/02

(52) U.S. Cl. 600/439; 601/3

(57) ABSTRACT

The present invention relates to a precise blood vessel positioning and high-intensity focused ultrasound ablator by use of one ultrasound transducer.

### FIELD OF THE INVENTION

The present invention relates to a system that utilizes one therapeutic ultrasound transducer to be used for both blood vessel positioning diagnosis and high-intensity focused ultrasound (HIFU) ablation therapy.

### BACKGROUND OF THE INVENTION

According to recent basic and clinical research, HIFU ablation is a highly potent new cancer therapeutic method. Its main advantage is that it is neither invasive nor radioactive, so that it may be repeatedly administered without widespread side effects. However, there are still many problems to be solved before HIFU becoming a mature and widely accepted treatment option. For example, due to the fact that a single HIFU ablation focal spot is only several millimeters in diameter, it takes thousands of focal spots to cover the area of a whole tumor, taking too much time and increasing the risk for patients. Since tumor cells require great amounts of nutrients and oxygen because of their high metabolism and rapid growth, an ampler blood flow, supplied by one or more “supply vessels,” is often observed around tumor tissue, compared with normal tissue. Therefore, if the supply brought by these vessels is to be effectively cut off, the resulting death of tumor tissue may greatly shorten the HIFU operation time.

In recent years, a common but invasive approach for liver cancer treatment is transcatheter arterial embolization (TAE), which interdicts the blood flow to tumor cells. This is based on the fact that the liver has two channels for blood supply: the hepatic artery and the hepatic portal vein; the former is responsible for only one fourth of total liver blood supply while the latter is responsible for three fourth. Moreover, liver tumors rely solely on the hepatic artery for blood. By using a plug to block the supply vessel, cancer tissue will die from ischemia while normal liver tissue will survive. Therefore this method may be used to selectively target cancer cells.

HIFU has also been employed to stop blood flow by coagulation necrosis and thrombosis of the target vessels. (T. Ishikawa, T. Okai, K. Sasaki, S. Umemura, R. Fujiwara, M. Kushima, M. Ichihara, and K. Ichizuka, “Functional and histological changes in rat femoral arteries by HIFU exposure,” Ultrasound Med Biol, vol. 29, pp. 1471-7, 2003., K. Ichizuka, S. Ando, M. Ichihara, T. Ishikawa, N. Uchiyama, K. Sasaki, S. Umemura, R. Matsuoka, A. Sekizawa, T. Okai, T. Akabane, and M. Kushima, “Application of high-intensity focused ultrasound for umbilical artery occlusion in a rabbit model,” Ultrasound Obstet Gynecol, vol. 30, pp. 47-51, 2007., M. Ichihara, K. Sasaki, S. Umemura, M. Kushima, and T. Okai, “Blood flow occlusion via ultrasound image-guided high-intensity focused ultrasound and its effect on tissue perfusion,” Ultrasound Med Biol, vol. 33, pp. 452-9, 2007., K. Hynynen, V. Colucci, A. Chung, and F. Jolesz, “Noninvasive arterial occlusion using MRI-guided focused ultrasound,” Ultrasound Med. Biol., vol. 22, pp. 1071-1077, 1996.) Ablating vessels using HIFU requires the cooperation of at least two systems, one is the diagnostic system for the positioning of blood vessels and post-treatment evaluation; the other is the therapeutic system for the ablation treatment. The present invention, which is based on similar rationales, provides a novel device that integrates both diagnostic and therapeutic functions, and does not require complex ducting techniques to block blood vessels for therapeutic purposes. This present invention is also less cumbersome and less expensive, and may resolve the difficulty to obtain the same focus when using two different systems.

### BRIEF DESCRIPTION OF THE DRAWINGS

FIG. 1 shows the supply vessel of the liver tumor is about 1.7-2.4 mm in diameter in this photograph of a hepatic artery.

FIG. 2 shows the assembly of the ultrasonic system.

FIG. 3 shows the schematic diagram of the ultrasound transducer module.

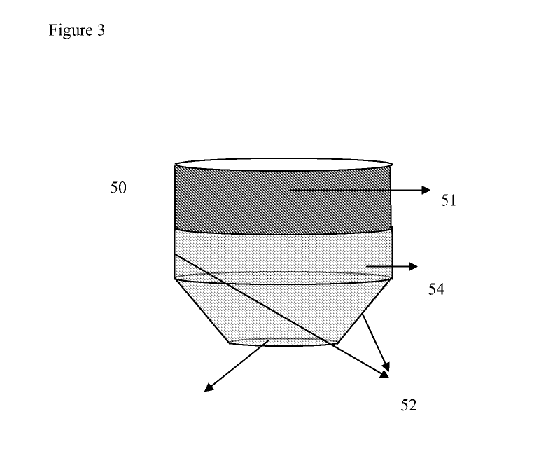

FIG. 4: shows blood Doppler frequency difference by a blood mimetic. (A) When there is no flow, the Doppler frequency difference is 0, and only the 0 Hz basal signal is observed; (B) Under conditions of 2 mm diameter and 16 cm/s flow, a notable Doppler frequency difference appears at the negative direction (away from the HIFU transducer). The horizontal axis is the frequency difference; the vertical axis is the adjusted amplitude.

FIG. 5 shows the result of ablating a blood vessel in a transparent mimetic after positioning with Doppler frequency difference. The focal spot produced by ablation (white area on the tube) is of the same position as that of the actual tube.

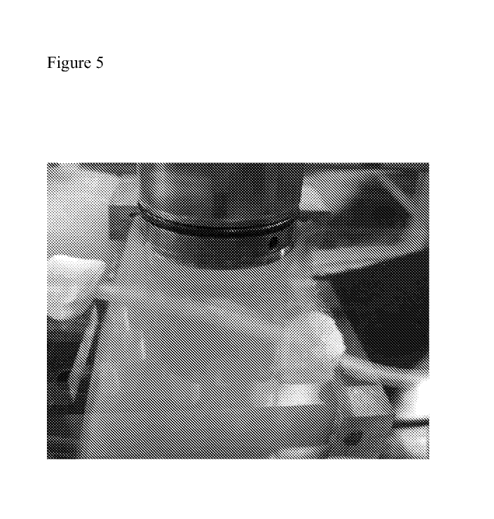

### SUMMARY OF THE INVENTION

The present invention discloses an ultrasonic diagnostic and therapeutic system, which utilizes high-intensity focused ultrasound for ablating blood vessels and blocking blood flow, comprises an ultrasound transducer module; a diplexer module; a diagnostic processor and a therapeutic processor.

The present invention also discloses a method for ablating selected vascular regions to block the blood flow by using the ultrasonic diagnostic and therapeutic system.

### DETAILED DESCRIPTION OF THE INVENTION

The present invention establishes a system that utilizes one therapeutic ultrasound transducer to be used for both blood vessel positioning diagnosis and high-intensity focused ultrasound (HIFU) ablation therapy. This system may be operated under low energy mode (diagnostic mode) prior to therapy, using the Doppler signal (blood flow signal change) generated by its ultrasound transducer to verify the location of the target vessel and position the device for ablation. The system may then be switched to high energy mode (therapeutic mode) for blood vessel ablation. During the ablation process, the system may be rapidly and repeatedly switched to low energy diagnostic mode, using the same Doppler signal to evaluate whether the position of the target has drifted away. The low energy diagnostic mode may also be used to analyze the degree of interdiction after the blood vessel has been seared, so that the operator can decide whether a switch back to therapeutic mode is required for further ablation.

The present invention discloses an ultrasonic diagnostic and therapeutic system (10), which utilizes high-intensity focused ultrasound for ablating blood vessels and blocking blood flow, comprises:

An ultrasound transducer module (50), for diagnosis and therapy, comprising a focused ultrasound transducer (51) and a plastic device (52);

A diplexer module (40) coupled to the ultrasound transducer module (50), for transmitting diagnostic ultrasound, receiving echo signals (Doppler signals) from blood flow in response to the transmitted ultrasound and delivering therapeutic ultrasound;

A diagnostic processor (20) coupled to the diplexer module (40), for producing diagnostic control signals and responsive to the echo signals for identifying a location of blood flow in real time; and

A therapeutic processor (30), coupled to the diplexer module (40), for producing therapeutic control signals.

The present focused ultrasound transducer (51) sustains high energy input and transmits high-intensity ultrasound for ablation therapy.

In addition, the present plastic device (52) is mounted at the tip of the focused ultrasound transducer (51), and switched to different types to adjust the distance between the skin and the focused ultrasound transducer (51).

The present plastic device (52) also comprises an ultrasound-permeable membrane (53) that enables the plastic device (52) to be filled with ultrasound-conductible media (54).

The present diplexer module (40) receiving signals from the diagnostic processor (20) under low energy power levels controls the transmission of ultrasound through the ultrasound transducer module (50) to the identified area. In addition, this diplexer module (40) receiving signals from the therapeutic processor (30) under high energy power levels is also operable to deliver high-intensity focused ultrasound through the ultrasound transducer module (50) to the target area.

The present diagnostic processor (20) comprises a Doppler signal processor responsive to the receipt of Doppler echo signals (Doppler frequency difference) for identifying and verifying a location of blood motion and monitoring the therapeutic efficacy. However, the Doppler signal processor is responsive to the echo signal resulting from the flow velocity.

The present system may further comprise an audible emitter (60) responsive to the diagnostic processor (20), which is actuated indicating the location of blood flow and guiding the placement of the ultrasound transducer module (50) through varying loudness and frequencies of sound, enables the operator to know the change in blood signal in real time.

The present invention also discloses a method for ablating selected vascular regions to block the blood flow by using the ultrasonic diagnostic and therapeutic system, comprising the steps of: (1) affixing an ultrasound transducer module to a selected position; (2) identifying the appropriate treatment site to be heated with a ultrasound transducer module operated at diagnostic power levels (low energy levels); (3) using high intensity focused ultrasound to heat the identified site of blood flow with a ultrasound transducer operated at therapeutic power levels (high energy levels); (4) verifying the position and monitoring the efficacy with a ultrasound transducer operated at diagnostic power levels during heating; and (5) repeating the step of heating, if required,

wherein the steps of identifying, verifying and monitoring are performed with a focused ultrasound transducer and the step of heating are also performed with the same focused ultrasound transducer.

In addition, the method further comprise the step of analyzing the image to identify and select the at least one blood vessel by using 2D or 3D imaging ultrasound prior to entire treatment process.

The steps of identifying, verifying and monitoring are using voice feedback transformed from Doppler echo signals resulting from the blood flow of the target.

### EXAMPLES

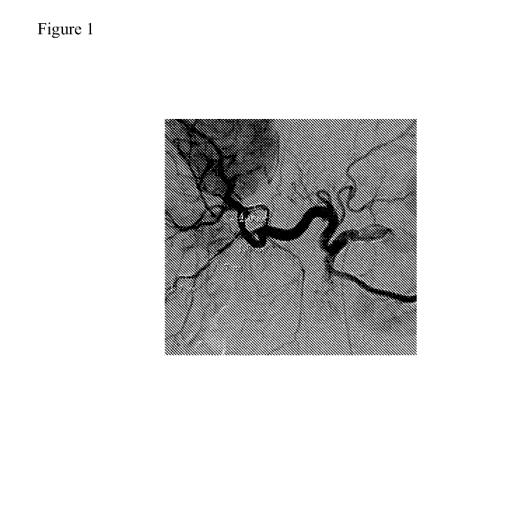

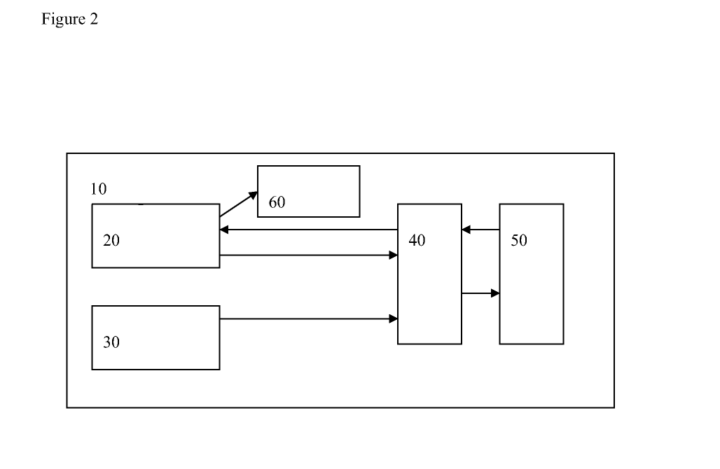

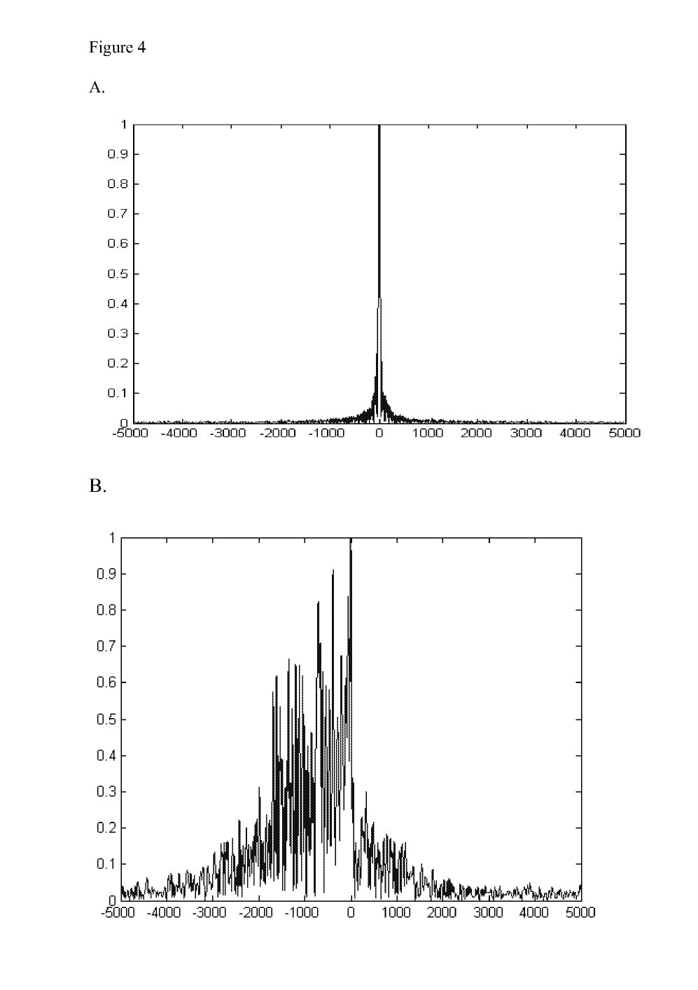

The key to this device was whether the HIFU therapeutic transducer used for ablation focused ultrasound transducer (51) functioned under mode, and whether it had enough resolving power to resolve the Doppler signal generated in the supply vessels of tumors 1-3 mm in diameter (liver cancer was used as an example here, as in FIG. 1.).
1. The ablation device was assembled as in FIG. 2. The most important device was a diplexer module that could separate the ultrasonic signal scattered from the target, using it as a basis for the calculation of the Doppler signal. The therapeutic processor (20) had a fast A/D function (>50 MHz resolving power) that could calculate the Doppler frequency difference in real time and an audible emitter (60) that emitted sounds of varying loudness and frequencies reflecting the Doppler frequency and magnitude differences. This sound could be used as a feedback for the operator's position and therapeutic efficacy.
2. The plastic device (52) at the tip of the focused ultrasound transducer (51) had different heights. By changing this plastic device, the distance between the transducer and the skin (a skin mimetic was used here.) could be adjusted. The higher the height of this plastic device, the closer the ablation focus was to the skin.
3. By using the ultrasound transducer module (50) to detect the Doppler signal arising from the flowing fluid inside a 2 mm inner diameter silicon tube and subsequently analyzing it with the therapeutic processor (20), it was shown, as in FIG. 4, that the ultrasound transducer module (50) could be used to clearly detect the Doppler signal.
4. By positioning the ultrasound transducer module (50) at the spot of the strongest Doppler signal (There would be a frequency difference as long as the focus was on the tube, but the magnitude was the strongest at the center, where the blood flow was the fastest.), by emitting a therapeutic mode signal by the therapeutic processor (30), and by ablating the vessel with the ultrasound transducer module (50), it was shown that the ablation focal spot was on the middle of the tube, as expected.

What is claimed is:

1. A ultrasonic diagnostic and therapeutic system, which utilizes high-intensity focused ultrasound for ablating blood vessels and blocking blood flow, comprising:
(1) an ultrasound transducer module, for diagnosis and therapy, comprising a focused ultrasound transducer and a plastic device;
(2) a diplexer module coupled to the ultrasound transducer module, for transmitting diagnostic ultrasound, receiving echo signals (Doppler signals) from blood flow in response to the transmitted ultrasound and delivering therapeutic ultrasound;
(3) a diagnostic processor coupled to the diplexer module, for producing diagnostic control signals and responsive to the echo signals for identifying a location of blood flow in real time; and
(4) a therapeutic processor coupled to the diplexer module, for producing therapeutic control signals.

2. The ultrasonic diagnostic and therapeutic system according to claim 1, wherein the focused ultrasound transducer sustains high energy input and transmits high-intensity ultrasound for ablation therapy.

3. The ultrasonic diagnostic and therapeutic system according to claim 1, wherein the plastic device is mounted at the tip of the focused ultrasound transducer, and switched to different types to adjust the distance between the skin and the focused ultrasound transducer.

4. The ultrasonic diagnostic and therapeutic system according to claim 3, wherein the plastic device comprises an ultrasound-permeable membrane that enables the plastic device to be filled with ultrasound-conductible media.

5. The ultrasonic diagnostic and therapeutic system according to claim 1, wherein the diplexer module receiving signals from the diagnostic processor under low energy power levels controls the transmission of ultrasound through the ultrasound transducer module to the identified area.

6. The ultrasonic diagnostic and therapeutic system according to claim 1, wherein the diplexer module receiving signals from the therapeutic processor under high energy power levels is operable to deliver high intensity focused ultrasound through the ultrasound transducer to the target area.

7. The ultrasonic diagnostic and therapeutic system according to claim 1, wherein the diagnostic processor comprises a Doppler signal processor responsive to the receipt of Doppler echo signals (Doppler frequency difference) for identifying and verifying a location of blood motion and monitoring the therapeutic efficacy.

8. The ultrasonic diagnostic and therapeutic system according to claim 7, wherein the Doppler signal processor is responsive to the echo signal resulting from the flow velocity.

9. The ultrasonic diagnostic and therapeutic system according to claim 1, further comprising an audible emitter responsive to the diagnostic processor which is actuated indicating the location of blood flow and guiding the placement of the ultrasound transducer through varying loudness and frequencies.

10. A method for ablating selected vascular regions to block the blood flow by using the ultrasonic diagnostic and therapeutic system according to claim 1, comprising the steps of:
(1) affixing an ultrasound transducer module to a selected position;
(2) identifying the appropriate treatment site to be heated with a ultrasound transducer module operated at diagnostic power levels (low energy levels);
(3) using high intensity focused ultrasound to heat the identified site of blood flow with a ultrasound transducer operated at therapeutic power levels (high energy levels);
(4) verifying the position and monitoring the efficacy with a ultrasound transducer operated at diagnostic power levels during heating; and
(5) repeating the step of heating, if required,
wherein the steps of identifying, verifying and monitoring are performed with a focused ultrasound transducer and the step of heating are also performed with the same focused ultrasound transducer.

11. The method according to claim 10, if required, further comprising the step of analyzing the image to identify and select the at least one blood vessel by using 2D or 3D imaging ultrasound prior to entire treatment process.

12. The method according claim 10, wherein the steps of identifying, verifying and monitoring are using voice feedback transformed from Doppler echo signals resulting from the blood flow of the target.
